(19) United States
(12) Patent Application Publication
(10) Pub. No.: US 2012/0123362 A1
(43) Pub. Date: May 17, 2012

(54) UROSTOMY BAG COVER SYSTEMS

(76) Inventors: Mary Ann Burroughs, Gambrills, MD (US); Carolyn S. Burroughs Hicks, Bowie, MD (US)

(21) Appl. No.: 13/295,076

(22) Filed: Nov. 13, 2011

Related U.S. Application Data
(60) Provisional application No. 61/413,558, filed on Nov. 15, 2010.

(51) Int. Cl. A61F 5/445

(52) U.S. Cl. 604/337

(57) ABSTRACT

Urostomy bag cover system comprising: an ostomy bag cover; a wafer baseplate access hole; and an elastic retainer for retaining the ostomy bag cover about the ostomy bag. The wafer baseplate access hole provides access to a wafer baseplate located in the ostomy bag. The ostomy bag cover provides a non-obstructive removeable-covering for the ostomy bag, with the ostomy bag providing for the collection of waste from a surgically diverted biological system. The ostomy bag comprises a sealable collection pouch for containing waste bodily fluids and is removably couplable to a digestive system or a urinary system. The ostomy bag cover comprises at least one cotton/spandex blend decorative fabric with the decorative fabric acting as a concealer. Decorative fabric of ostomy bag cover may match a garment-fabric-appearance.

### CROSS-REFERENCE TO RELATED APPLICATION

The present application is related to and claims priority from prior provisional application Ser. No. 61/413,558, filed Nov. 15, 2010 which application is incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

1. Field of the Invention

The present invention relates generally to the field of medical appliance covers and more specifically relates to an ostomy bag cover configured specifically to conceal an ostomy pouch.

2. Description of the Related Art

Every day, thousands of consumers across the country undergo stoma surgical procedures resulting in a permanent or temporary ostomy. An ostomy is an artificial opening in the abdomen through which a portion of the patient's bowel is pulled, thus allowing solid or fluid waste matter to exit the body without passing through the rectum or urethra. This surgery is performed for a variety of reasons including abdominal obstruction, diseases such as cancer, Chron's disease and ulcerative colitis, as well as a result of trauma and congenital defects. Ostomy procedures are broken into three major categories: urostomy; ileostomy; and colostomy. A urostomy is a procedure where the ureters are detached from the bladder and joined to a small section of the small intestine to form a stoma. Urine is then excreted through this stoma and collected in an external urostomy pouch.

In medicine, a stoma is an opening either natural or surgically created (artificial), which connects a portion of the body cavity to the outside environment. In anatomy, a natural stoma is any opening in the body, such as the mouth. Any hollow organ can be manipulated into an artificial stoma as necessary. This includes the esophagus, stomach, duodenum, ileum, colon, pleural cavity, ureters, urinary bladder, and kidney pelvis. A urostomy is a stoma (artificial opening) for the urinary system. A urostomy is made in cases where long-term drainage of urine through the bladder and urethra is not possible, for example after extensive surgery or in case of obstruction.

Ostomy pouching systems may include a one-piece or two-piece system, each including a wafer (faceplate/flange) and a collection pouch. The pouch (one-piece or two-piece) attaches to the abdomen by the wafer and is fitted over and around the stoma to collect the diverted output, either stool or urine. This barrier is designed to protect the skin from the stoma output and to be as gentle on the skin as possible.

While stoma surgery can be a lifesaving and necessary procedure, adapting to life with an ostomy pouch may prove difficult. Learning the proper care and maintenance for both a stoma and an ostomy pouch can be somewhat overwhelming during the first few days after surgery. Over time however, most ostomy patients find that they adapt to this new routine and can, in many cases, resume their normal lifestyle. Unfortunately however, because these devices can be bulky, difficult to conceal, and generally aesthetically unappealing, many consumers find themselves wearing loose, unflattering clothing, simply in order to hide their ostomy bag from view. For children and fashion conscious teens, this problem can be particularly challenging, as an ostomy bag can set them apart from their peers, often making them feel “different” and self conscious. It is desirable that ostomy bags be concealed from detection of their presence.

Various attempts have been made to solve problems found in urostomy bag art. Among these are found in: U.S. Pat. No. 5,607,412 to Brown, U.S. Design Pat. No. D245,119 to Harris, U.S. Design Pat. No. D470,586 to Felstet, and U.S. Design Pat. No. D484,240 to Lyons. This prior art is representative of medical appliance covers and these patents, taken either singly or in combination, is not seen to describe the invention as claimed.

Ideally, a urostomy bag cover system should be safe, user-friendly and, yet would operate reliably and be manufactured at a modest expense. Thus, a need exists for reliable urostomy bag covers systems to avoid the above-mentioned problems.

### BRIEF SUMMARY OF THE INVENTION

In view of the foregoing disadvantages inherent in the known medical appliance art, the present invention provides a novel urostomy bag cover configured specifically to conceal an ostomy pouch. The general purpose of the present invention, which will be described subsequently in greater detail, is to provide an ostomy bag cover configured specifically to conceal an ostomy pouch.

Urostomy bag covers systems are disclosed herein in a preferred embodiment preferably comprising: an ostomy bag cover; having a wafer baseplate access hole; and an elastic retainer for retaining the ostomy bag cover about the ostomy bag and the wafer baseplate access hole. The wafer baseplate access hole is designed to provide access to the wafer baseplate located in the ostomy bag.

The ostomy bag cover provides a non-obstructive removeable-covering for the ostomy bag, with the ostomy bag providing for the collection of waste from a surgically diverted biological system. The ostomy bag comprises a sealable collection pouch for sealably-containing waste bodily fluids and is removably couplable to a digestive system or a urinary system.

The ostomy bag in the preferred embodiment has a top wall; a bottom wall; a proximate end wall; a distal end wall; a left side wall; and a right side wall. The top wall, the bottom wall, the proximate end wall, the distal end wall, the left side wall; and the right side wall define an inner volume providing the ostomy bag cover for suitably covering an ostomy bag with a top wall; a bottom wall; a proximate end wall; a distal end wall; a left side wall; and a right side wall.

The ostomy bag cover for use with an open-end ostomy bag further comprises a resealable tube access hole. The resealable tube access hole is located on the distal end wall of the ostomy bag cover wherein a resealable tube is located therethrough the resealable tube access hole and the resealable tube access hole is usable to provide egress of the waste bodily fluids.

The ostomy bag cover is reuseable and machine-washable in preferred embodiments. The ostomy bag cover preferably comprises at least one decorative fabric, which acts as a concealer. The decorative fabric of the ostomy bag cover preferably matches a garment-fabric-appearance. Further, the fabric may be a cotton/spandex blend. The ostomy bag cover provides a cushioner and comprises about ¼ inch thick padding. The ostomy bag cover provides a sanitary barrier between the ostomy bag and a contact-skin-region of the user-wearer. Also, the ostomy bag cover provides a germ and bacteria resistant surface.

A kit is embodied herein for the urostomy bag cover systems comprising: a plurality of decorative ostomy bag covers; and a set of user instructions.

A method of using an ostomy bag cover system is disclosed herein preferably comprising the steps of: purchasing a ostomy bag cover in a user-chosen design to fit the dimensions of a ostomy bag; applying the ostomy bag cover over the ostomy bag via elastic retainers; sealing the ostomy bag to a user-wearer; concealing the ostomy bag; removing the ostomy bag cover from the ostomy bag once filled with waste bodily fluids of the user-wearer; draining the ostomy bag with the ostomy bag cover remaining about the ostomy bag; and washing the ostomy bag cover when removed from the ostomy bag.

The present invention holds significant improvements and serves as an urostomy bag cover systems. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, urostomy bag cover systems constructed and operative according to the teachings of the present invention.

FIG. 1 shows a perspective view illustrating urostomy bag cover systems in an ‘in-use’ position according to an embodiment of the present invention

FIG. 2 is a perspective view illustrating an ostomy bag cover covering an ostomy bag having an open-end according to an embodiment of the present invention.

FIG. 3 is a perspective view illustrating the ostomy bag cover and open-end version of the ostomy bag (dashed lines) according to an embodiment of the present invention.

FIG. 4 is a perspective view illustrating another view of an ostomy bag cover for use with an ostomy bag having a closed end according to an embodiment of the present invention.

FIG. 5 is a flowchart illustrating a method of use according to an embodiment of the present invention of FIGS. 1-4.

The various embodiments of the present invention will hereinafter be described in conjunction with the appended drawings, wherein like designations denote like elements.

### DETAILED DESCRIPTION

As discussed above, embodiments of the present invention relate to medical appliance cover art, the present invention provides a novel urostomy bag cover configured specifically to conceal an ostomy pouch. The general purpose of the present invention, which will be described subsequently in greater detail, is to provide an ostomy bag cover configured specifically to conceal an ostomy pouch.

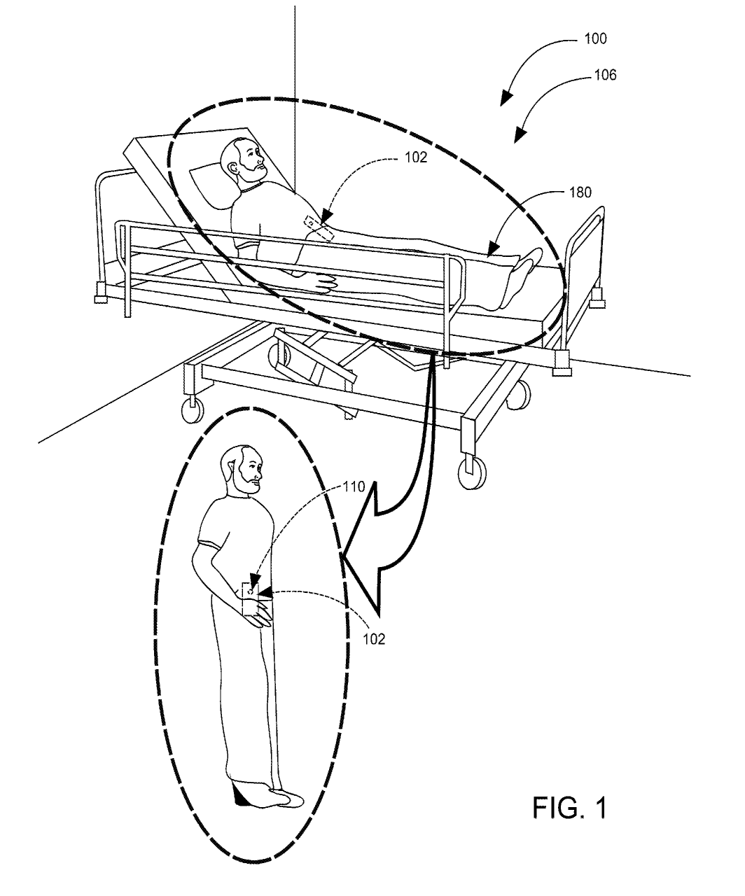

Referring to the drawings by numerals of reference there is shown in FIG. 1, urostomy bag cover systems 100 in in-use condition 106 according to an embodiment of the present invention.

Urostomy bag cover systems 100 preferably comprises ostomy bag cover 102; wafer baseplate access hole 120; and elastic retainer 130 for retaining ostomy bag cover 102 about ostomy bag 150 and wafer baseplate access hole 110. Wafer baseplate access hole 120 preferably provides access to wafer baseplate 110 located in ostomy bag 150.

Ostomy bag cover(s) 102 provide a means to assist user-wearer 180 who have undergone ostomy surgery with a safe, comfortable and discreet means of concealing ostomy bag 150, thus enabling user-wearer 180 to enjoy a more carefree lifestyle as shown in ‘in-use’ condition 106 of FIG. 1. Further, ostomy bag cover 102 provides a non-obstructive removeable-covering for ostomy bag 150, with ostomy bag 150 providing for the collection of waste from a surgically diverted biological system. Ostomy bag 150 preferably comprises sealable collection pouch 154 for containing waste bodily fluids and is removably couplable to a digestive system or a urinary system.

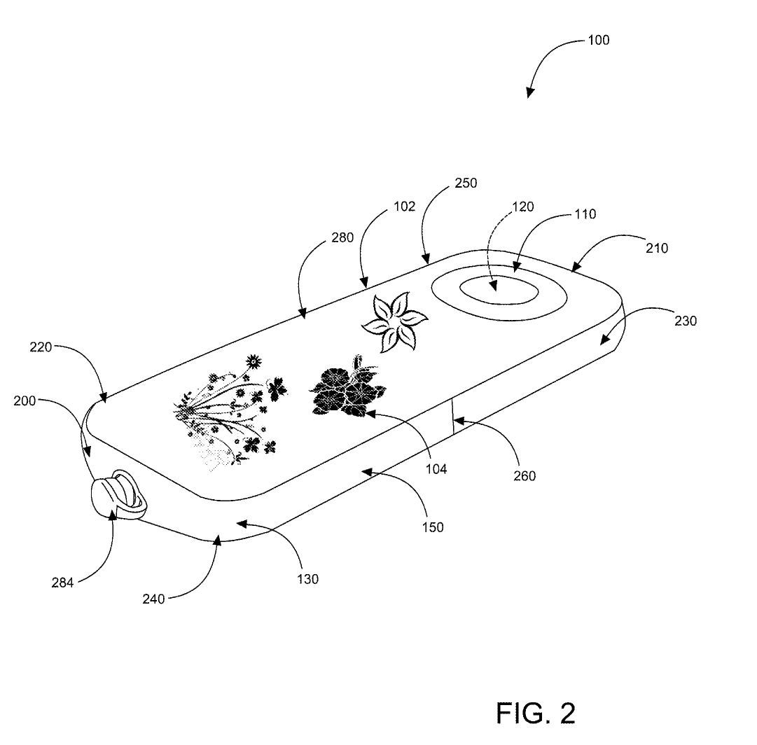

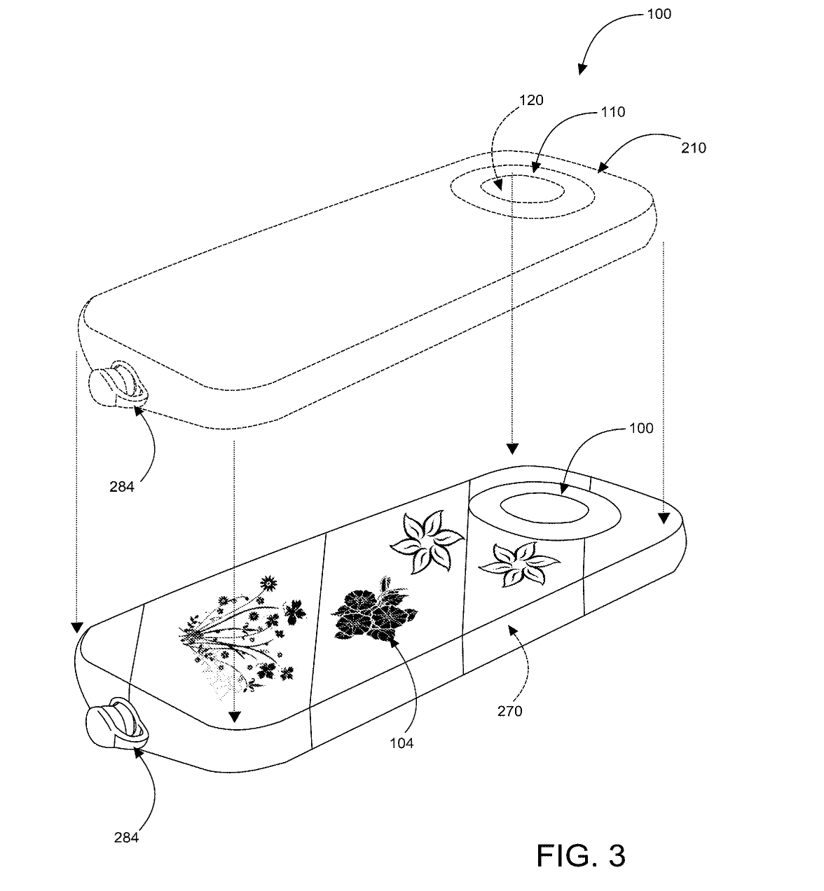

Referring now to FIGS. 2-3 is a perspective view illustrating open-end ostomy bag 200 of the urostomy bag cover systems 100 according to an embodiment of the present invention.

Ostomy bag cover(s) 102 includes top wall 210; bottom wall 220; proximate end wall 230; distal end wall 240; left side wall 250; and right side wall 260. Top wall 210; bottom wall 220; proximate end wall 230; distal end wall 240; left side wall 250; and right side wall 260 preferably define inner volume 270 providing ostomy bag cover 102 such that it can suitably cover ostomy bag 150. Further, ostomy bag cover 102 may be re-useable or disposable. When ostomy bag cover 102 is in reusable form, it may be machine-washable and/or hand-washable. When ostomy bag cover 102 is disposable, after a designated amount of time, ostomy bag cover 102 is required to be discarded for a new ostomy bag cover 102.

Ostomy bag cover 102 preferably comprises about ¼ inch thick padding. This ¼ inch thick padding of ostomy bag cover 102 provides a cushioner between ostomy bag 150 and a contact-skin-region of user-wearer 180. Ostomy bag cover 102 preferably additionally provides a sanitary bather between ostomy bag 150 and the contact-skin-region of user-wearer 180. Ostomy bag cover 102 may also provide a germ and bacteria resistant surface.

Additionally, ostomy bag cover 102 preferably comprises at least one decorative fabric 104, with decorative fabric 104 acting as a concealer as shown best in FIG. 2. Decorative fabric 104 may be a cotton/spandex blend. Decorative fabric 104 of ostomy bag cover 102 may match a garment-fabric-appearance. Decorative fabric 104 may be made in a variety of vibrant colors, as well as in a host of attractive patterns and printed designs. Upon reading this specification, it should be appreciated that, under appropriate circumstances, considering such issues as user preferences, design preference, structural requirements, marketing preferences, cost, available materials, technological advances, etc., other decorative fabric material arrangements such as, for example, cloth, various types of fabrics, rubbers, etc., may be sufficient.

Ostomy bag cover 102 is retained about ostomy bag 150 and wafer baseplate access hole 120 via elastic retainer 130. Generally oval in shape, ostomy bag cover 102 may be custom fitted to an existing ostomy bag 150 by way of elastic retainer 130 which includes a deformable (able to stretch) elastic band built into cotton/spandex blend of decorative fabric 104 of ostomy bag cover 102 as shown best in FIG. 3.

Ostomy bag 150 may include a one-piece or two-piece system, each including a wafer baseplate 110 and collection pouch 154. Wafer baseplate access hole 110 is preferably located on top wall 210 of ostomy bag cover 102. Collection pouch 154 of ostomy bag 150 preferably attaches to an abdomen of user 180 by wafer baseplate 110 and is fitted over and around a stoma to collect the diverted output, either stool or urine. Wafer baseplate 110 is designed to protect skin of user 180 from the stoma output and to be as gentle and neutral on the skin as possible.

Wafer baseplate 110 is preferably designed to protect the skin from the stoma output and to be as neutral to the skin as possible with wafer baseplate access hole 120 preferably accommodating the stoma of user-wearer 180. Wafer baseplate 110 may be manufactured using pectin or similar organic material and may be available in a wide variety of sizes to accommodate a person's particular anatomy. Wafer baseplate access hole 120 of wafer baseplate 110 preferably must be the correct size to accommodate the stoma of user 180 while protecting the skin from contact with waste such as bodily fluids (urine and feces). The methods for sizing wafer baseplate access hole 120 may vary depending on the type of wafer/baseplate 110 with some pre-cut sizes available, however most users may customize the opening using scissors.

Additionally, the method of attachment to wafer baseplate 110 to a stoma on user 180 varies between manufactures and includes permanent (one-piece), press-on/click (“Tupperware” type), turning locking rings and “sticky” adhesive mounts. The two-piece arrangement allows pouches to be exchanged without removing wafer baseplate 110; for example, however some people prefer to temporarily switch to a “mini-pouch” for swimming, intimate and other short-term activities. It should be noted that mini-pouches are suitable for minimum usage only.

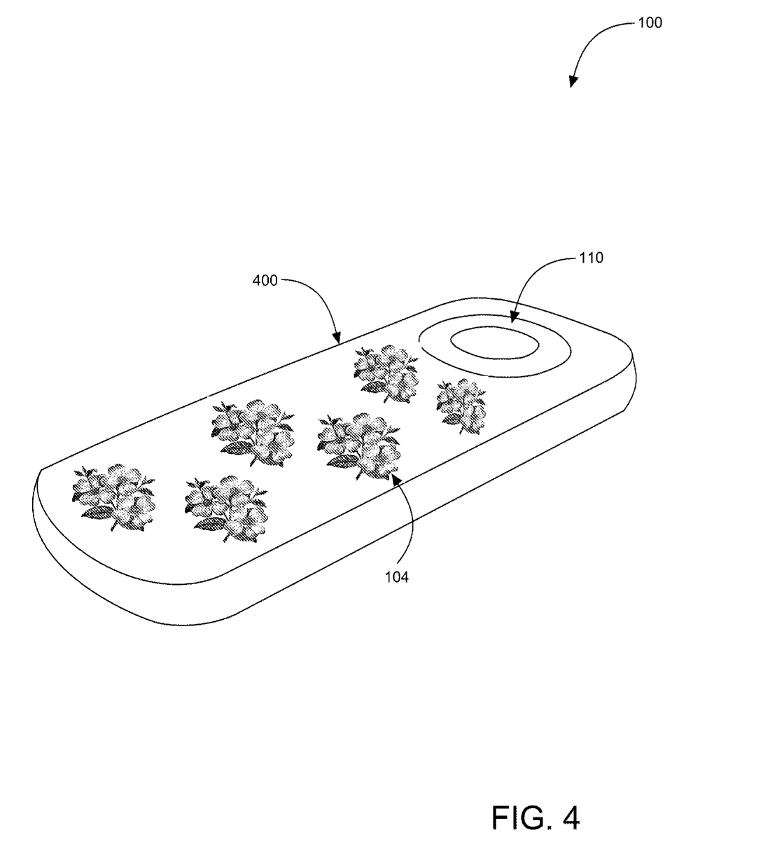

Ostomy bag 150 may be broken down into two basic types: open-end ostomy bag (drainable) 200 as show in FIGS. 2 & 3 and closed-end ostomy bag (disposable) 400 as shown in FIG. 4. Open-end ostomy bag 280 comprises resealable tube access hole 284 located preferably on distal end wall 240 of ostomy bag cover 102. A resealable tube (not shown) is located therethrough resealable tube access hole 284 and resealable tube access hole 284 is usable to provide egress of waste bodily fluids. When collection pouch 154 on open-end ostomy bag 200 is full, resealable tube access hole 284 may be opened with the waste bodily fluids being drained and disposed of into a toilet.

Referring now to FIG. 4 showing a perspective view illustrating closed-end ostomy bag 400 of the urostomy bag cover systems 100 according to an embodiment of the present invention.

Closed-end ostomy bag 400 preferably comprises collection pouch 154 that is disposable. Closed-end ostomy bag 400 may be removed and replaced with a new closed-end ostomy bag 400 once collection pouch 154 is full. For maximum hygiene and to reduce risk of infection, closed-end ostomy bag 400 should be changed every twenty-four hours or as per physician's orders. A drawback of using closed-end ostomy bag 400 is the increased chance of extreme damage to the skin area under the adhesive due to the frequency of removal and re-application of wafer baseplate 110 to user-wearer 180.

Urostomy bag cover systems 100 may be sold as kit 440 comprising a plurality of decorative ostomy bag covers 102; and a set of user instructions. Other decorative ostomy bag covers 102 may be included within kit 440 of varying sizes or the like for use with different versions. Urostomy bag cover systems 100 may be manufactured and provided for sale in a wide variety of sizes and shapes for a wide assortment of applications. Upon reading this specification, it should be appreciated that, under appropriate circumstances, considering such issues as design preference, user preferences, marketing preferences, cost, structural requirements, available materials, technological advances, etc., other kit contents or arrangements such as, for example, including more or less components, customized colors/designs, parts may be sold separately, etc., may be sufficient.

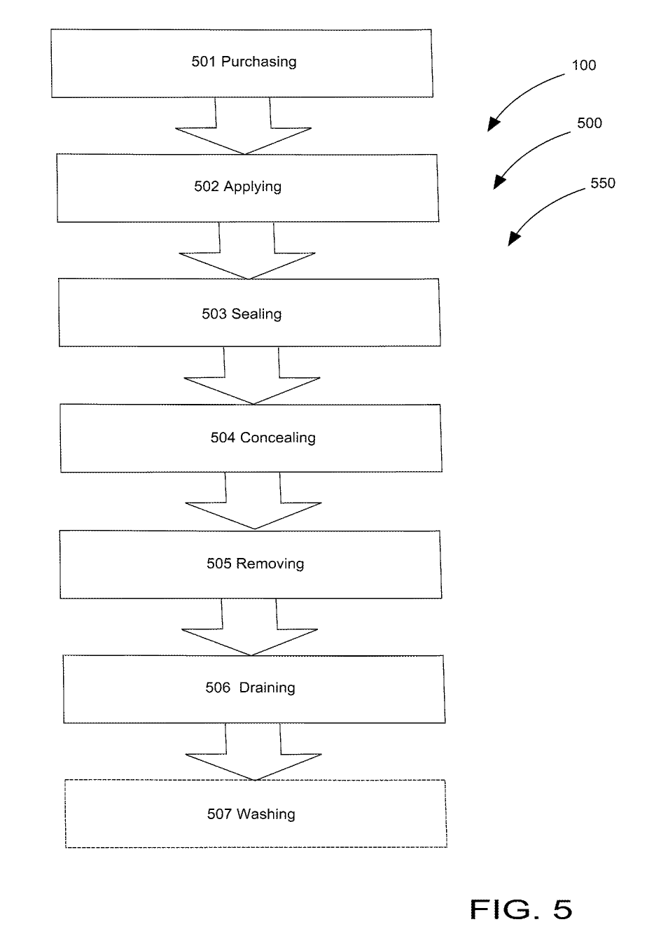

Referring now to FIG. 5, a flowchart 550 illustrating a method of using (method of use 500) urostomy bag cover system 100 according to an embodiment of the present invention of FIGS. 1-4.

A method of using (enabling method of use 500) urostomy bag cover system 100 preferably comprises the steps of: step one 501 purchasing ostomy bag cover 102 in a user-chosen design to fit the dimensions of ostomy bag 150; step two 502 applying ostomy bag cover 102 over ostomy bag 150 via elastic retainers 130; step three 503 sealing ostomy bag 150 to user-wearer 180; step four 504 concealing ostomy bag 150; step five 505 removing ostomy bag cover 102 from ostomy bag 150 once filled with waste bodily fluids of the user-wearer 180; step six 506 draining ostomy bag 150 with ostomy bag cover 102 remaining about ostomy bag 150; and step seven 507 washing ostomy bag cover 102 (once removed.)

It should be noted that step 507 is an optional step and may not be implemented in all cases. Optional steps of method 500 are illustrated using dotted lines in FIG. 5 so as to distinguish them from the other steps of method 500.

It should be noted that the steps described in the method of use can be carried out in many different orders according to user preference. The use of “step of” should not be interpreted as “step for”, in the claims herein and is not intended to invoke the provisions of 35 U.S.C. §112, ¶6. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. An ostomy bag cover system comprising:
an ostomy bag cover having;
a top wall;
a bottom wall;
a proximate end wall;
a distal end wall;
a left side wall; and
a right side wall;
an elastic retainer;
a wafer baseplate access hole located on said top wall of said ostomy bag cover;
wherein said said top wall, said bottom wall, said proximate end wall, said distal end wall, said left side wall; and said right side wall define an inner volume providing said ostomy bag cover for suitably covering an ostomy bag;
wherein said wafer baseplate access hole provides access to a wafer baseplate located in said ostomy bag;
wherein said wafer baseplate access hole accommodates a stoma of a user-wearer;
wherein said ostomy bag comprises a sealable collection pouch for containing waste bodily fluids;
wherein said elasticized retainer retains said ostomy bag cover about said ostomy bag and said wafer baseplate access hole; and
wherein said ostomy bag cover provides a non-obstructive removeable-covering for said ostomy bag, said ostomy bag providing for the collection of waste from a surgically diverted biological system.

2. The ostomy bag cover system of claim 1 further comprising a resealable tube access hole located on said distal end wall of said ostomy bag cover wherein a resealable tube is located therethrough said resealable tube access hole and said resealable tube access hole is usable to provide egress of said waste bodily fluids.

3. The ostomy bag cover system of claim 2 wherein said wherein said ostomy bag cover is for use with an open-end said ostomy bag.

4. The ostomy bag cover system of claim 1 wherein said wherein said ostomy bag cover is for use with an closed-end said ostomy bag.

5. The ostomy bag cover system of claim 1 wherein said ostomy bag cover is reuseable.

6. The ostomy bag cover system of claim 2 wherein said ostomy bag cover is machine-washable.

7. The ostomy bag cover system of claim 6 wherein said ostomy bag cover provides a cushioner.

8. The ostomy bag cover system of claim 4 wherein said ostomy bag cover is disposable.

9. The ostomy bag cover system of claim 7 wherein said ostomy bag cover comprises at least one decorative fabric, said decorative fabric acting as a concealer.

10. The ostomy bag cover system of claim 9 wherein said fabric is a cotton/spandex blend.

11. The ostomy bag cover system of claim 7 wherein said ostomy bag cover comprises about ¼ inch thick padding.

12. The ostomy bag cover system of claim 1 wherein said ostomy bag cover provides a sanitary barrier between said ostomy bag and a contact-skin-region of said user-wearer.

13. The ostomy bag cover system of claim 1 wherein said ostomy bag cover provides a germ and bacteria resistant surface.

14. The ostomy bag cover system of claim 9 wherein said decorative fabric of said ostomy bag cover matches a garment-fabric-appearance.

15. The ostomy bag cover system of claim 1 wherein said ostomy bag is removably couplable to a digestive system.

16. The ostomy bag cover system of claim 1 wherein said ostomy bag is removably couplable to a urinary system.

17. An ostomy bag cover system comprising:
an ostomy bag cover having;
a top wall;
a bottom wall;
a proximate end wall;
a distal end wall;
a left side wall; and
a right side wall;
an elastic retainer;
a wafer baseplate access hole located on said top wall of said ostomy bag cover; and
a resealable tube access hole;
wherein said said top wall, said bottom wall, said proximate end wall, said distal end wall, said left side wall; and said right side wall define an inner volume providing said ostomy bag cover for suitably covering an ostomy bag;
wherein said wafer baseplate access hole provides access to a wafer baseplate located in said ostomy bag;
wherein said wafer baseplate access hole accommodates a stoma of a user-wearer;
wherein said ostomy bag comprises a sealable collection pouch for containing waste bodily fluids;
wherein said ostomy bag cover is for use with an open-end said ostomy bag;
wherein said ostomy bag cover is reuseable;
wherein said ostomy bag cover is machine-washable;
wherein said ostomy bag cover comprises at least one decorative fabric, said decorative fabric acting as a concealer;
wherein said fabric is a cotton/spandex blend;
wherein said decorative fabric of said ostomy bag cover matches a garment-fabric-appearance;
wherein said ostomy bag cover comprises about ¼ inch thick padding;
wherein said ostomy bag cover provides a sanitary barrier between said ostomy bag and a contact-skin-region of said user-wearer;
wherein said ostomy bag cover provides a germ and bacteria resistant surface;
wherein said ostomy bag is removably couplable to a waste bodily fluid system;
wherein said elasticized retainer retains said ostomy bag cover about said ostomy bag and said wafer baseplate access hole;
wherein said ostomy bag cover provides a cushioner;
wherein said ostomy bag cover provides a non-obstructive removeable-covering for said ostomy bag, said ostomy bag providing for the collection of waste from a surgically diverted biological system; and
wherein said resealable tube access hole located on said distal end wall of said ostomy bag cover wherein a resealable tube is located therethrough said resealable tube access hole and said resealable tube access hole is usable to provide egress of said waste bodily fluids.

18. The ostomy bag cover system of claim 17 further comprising a kit, wherein said kit comprises: a plurality of decorative said ostomy bag covers; and a set of user instructions.

19. A method of using an ostomy bag cover system comprising the steps of:
purchasing a ostomy bag cover in a user-chosen design to fit the dimensions of a ostomy bag;
applying said ostomy bag cover over said ostomy bag via elastic retainers;
sealing said ostomy bag to a user-wearer;
concealing said ostomy bag;
removing said ostomy bag cover from said ostomy bag once filled with waste bodily fluids of said user-wearer; and
draining said ostomy bag with said ostomy bag cover remaining about said ostomy bag.

20. The method of using an ostomy bag cover system of claim 19 further comprising the step of washing said ostomy bag cover.
